(19) United States
(12) Patent Application Publication
(10) Pub. No.: US 2010/0010535 A1
(43) Pub. Date: Jan. 14, 2010

(54) EMBOLIC PROTECTION DEVICE

(75) Inventor: Husnija Mujkanovic, Santa Clara, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/172,644

(22) Filed: Jul. 14, 2008

(51) Int. Cl. A61M 29/02

(52) U.S. Cl. 606/200

(57) ABSTRACT

The disclosure relates to an embolic filter deployment system and a method for deploying an embolic filter from the deployment system wherein the filter element and a containment element for the filter element are formed from a single piece of material.

### TECHNICAL FIELD

This disclosure relates generally to an embolic filter deployment system and a method for deploying an embolic filter from the deployment system.

### BACKGROUND

Human blood vessels often become occluded or blocked by plaque, thrombi, other deposits, or material that reduce the blood carrying capacity of the vessel. Should the blockage occur at a critical place in the circulatory system, serious and permanent injury, and even death, can occur. To prevent this, some form of medical intervention is usually performed when significant occlusion is detected.

Several procedures are now used to open these stenosed or occluded blood vessels in a patient caused by the deposit of plaque or other material on the walls of the blood vessels. Angioplasty, for example, is a widely known procedure wherein an inflatable balloon is introduced into the occluded region. The balloon is inflated, dilating the occlusion, and thereby increasing the intraluminal diameter.

Another procedure is atherectomy. During atherectomy, a catheter is inserted into a narrowed artery to remove the matter occluding or narrowing the artery, i.e., fatty material. The catheter includes a rotating blade or cutter disposed in the tip thereof Also located at the tip are an aperture and a balloon disposed on the opposite side of the catheter tip from the aperture. As the tip is placed in close proximity to the fatty material, the balloon is inflated to force the aperture into contact with the fatty material. When the blade is rotated, portions of the fatty material are shaved off and retained within the interior lumen of the catheter. This process is repeated until a sufficient amount of fatty material is removed and substantially normal blood flow is resumed.

In another procedure, stenosis within arteries and other blood vessels is treated by permanently or temporarily introducing a stent into the stenosed region to open the lumen of the vessel. The stent typically includes a substantially cylindrical tube or mesh sleeve made from such materials as stainless steel or nitinol. The design of the material permits the diameter of the stent to be radially expanded, while still providing sufficient rigidity such that the stent maintains its shape once it has been enlarged to a desired size.

Such percutaneous interventional procedures, i.e., angioplasty, atherectomy, and stenting, can dislodge material from the vessel walls. This dislodged material can enter the bloodstream. Some existing devices and technology use a filter for capturing the dislodged material from the bloodstream.

### SUMMARY

The present disclosure pertains to an embolic filter deployment apparatus which can be configured to be used in connection with an intravascular device. The apparatus can include a guide wire, a filter element associated with the guide wire, a containment element, and an actuation element, movable between a first position and a second position to release the filter from the containment element. The filter element and the containment element can be regions of a single sheet substantially formed from a single piece of material, or multiple pieces bound together to form a single sheet.

Additionally, an embolic filter can be deployed from a containment element, wherein the filter and containment element are formed from a single piece of material, or multiple pieces bound to form a single sheet. The method includes providing a structure or structures for joining the various parts of the containment element and an actuation element, movable between a first position and a second position such that when the actuation element is in the first position, the joining structures or elements hold the containment element in position about the filter and in the second position, the actuation element allows the joining structures or elements release the containment element thereby deploying the filter.

### BRIEF DESCRIPTION OF THE DRAWING

FIG. 1A illustrates an intermediate stage of the assembly of a filter assembly and

FIG, 1B illustrates the filter assembly as it is ready for delivery to the vessel to be protected.

FIG. 2 illustrates a combined filter sheet.

FIG. 3A illustrates another combined filter sheet. FIGS. 3B and 3C illustrate details of actuation element and the joining element of the sheet of FIG. 3A.

FIG. 4A illustrates another combined filter sheet including joining elements shown as mating fasteners. FIG. 4B is a detail of the mating fasteners in the engaged configuration.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

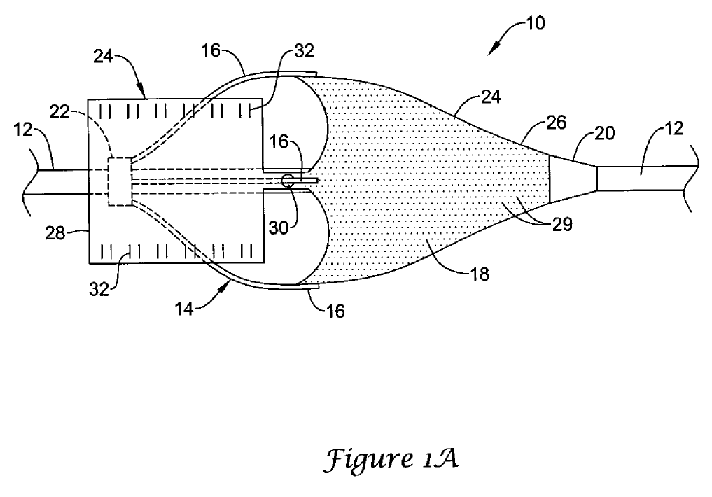

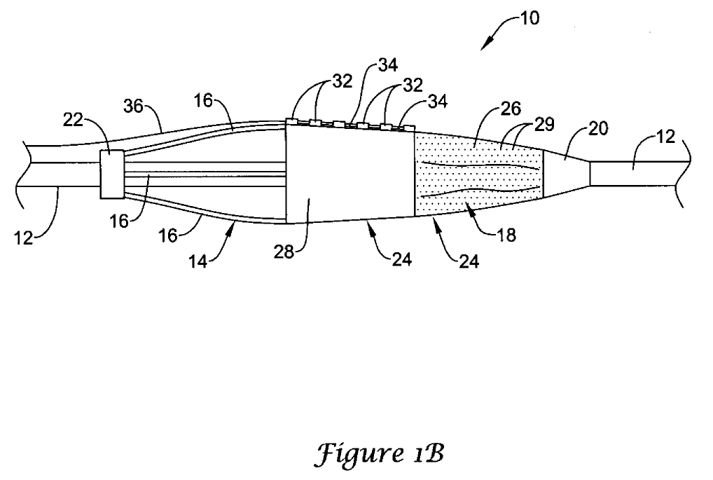

FIGS. 1A and 1B illustrate a filter assembly 10 disposed on a guide wire 12 Filter assembly 10 includes a filter frame 14 having a plurality of legs 16. Filter assembly 10 also includes the filter element 18 including a filter fabric or mesh having a plurality of small apertures. Filter element 18 can have a conical shape wherein a major opening is defined at the proximal end which can be supported by legs 16. The opposite end of filter element 18 can be attached to guide wire 18 at coupling 20. Filter element 18 can be fixably attached to guide wire 12 by adhesive, soddering, welding or other technique. Alternatively, coupling 20 can include a bushing such that filter element 18 can be slidably and or rotatably attached guide wire 12. Likewise, legs 16 of filter frame 14 can be attached to guide wire 12 by a coupling 22 by adhesive, soddering, welding or other technique. Alternatively, coupling 22 can be made to bushing to allow legs 16 and filter assembly 10 to rotate or slide on wire 12.

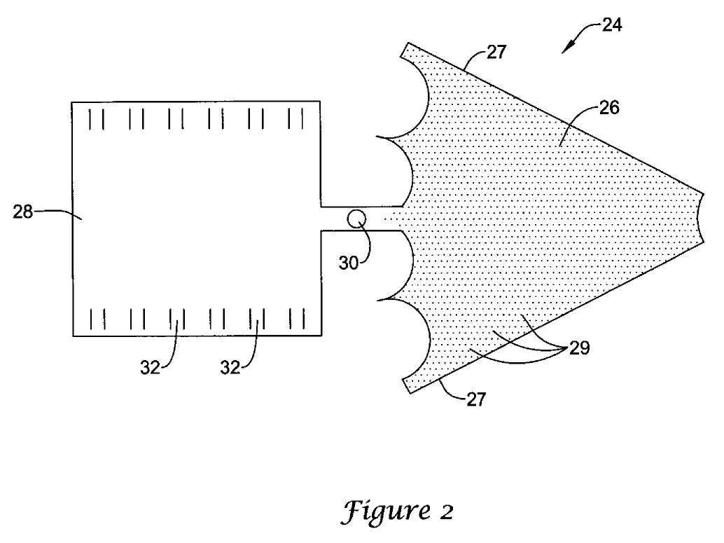

As shown in FIGS. 1A and 1B, and in more detail in FIG. 2, filter assembly 10 includes a combination filter sheet 24, having a filter element portion 26 and a containment element portion 28. In FIG. 1A, containment element portion 28 has been formed into a generally conically shaped filter element 18. Containment element portion 28 is shown in a pre-containment or pre-assembled position and includes an aperature 30 through which a leg 16 extends, and a plurality of joining slits 32. To complete the assembly, filter frame 14 and filter element 18 are compressed. Then containment element portion 28 is folded towards filter element 18, wrapped around filter element 18 and held in position by joining opposite sides of containment element portion 28 using joining slits 32.

As shown in FIG. 1B, containment element portion 28 has been folded over filter element portion 26 and joined at slits 32 by an actuation element 34 inserted through the slits on both sides of containment element 28. Actuation element 34 can be formed from a pin or cord inserted through slits 32 and include a cord 36 which extends approximately, to the proximal end of filter assembly 10, to allow a user to withdraw actuation filter 34 from slits 32. This will allow the sides of containment element 28 to separate. Filter element 18 can then expand aided by legs 16 which can be formed from elastic material such as nickel titanium alloy or nitinol. Once filter element 18 is expanded across a body lumen, such as a blood vessel, the major opening can, for example, receive a flow of blood including emboli to be captured by filter element 18 while blood passes through the small aperatures in filter element 18.

FIG. 2 shows a combination of filter sheet 24 as cut from a single sheet, prior to placement on filter frame 14. As shown in FIG. 2, filter element portion 26 is generally lying flat and includes opposite sides 27. When disposed on filter frame 14 and guide wire 12, filter element portion 26 is wrapped around guide wire 12 to form conically shaped filter element 18. Sides 27 are joined together by adhesive, welding or other method. Filter element portion 26 and containment element portion 28 may be cut from a single piece of polymeric sheet material or mesh in a single step to form combination filter sheet 24. As used herein, “single sheet” means only one sheet, however, the one sheet may be formed from one or more sheets bound together. Thus, combination filter sheet 24 can be cut from one sheet of material, inherently producing a combination filter sheet 24 in a “single sheet”. Alternatively, filter element portion 26 and containment element portion 28 may be formed from more than one sheet which are then bound together to form a “single sheet”. That is the more than one sheets are joined to become a “single” sheet which is a unitary combination of the more than one sheets.

The material used to form the combined filter sheet 24 may be, for example, laser cut to shape in the same process that cuts holes through which blood cells, but not emboli and other debris, may pass. The same operation also may form apertures or other structures which serve as joining slits or elements that, alone or in combination with an actuating element hold the filter element portion of the unitary filter assembly and containment element portion in a folded or otherwise compact configuration when actuation element 34 is in a first position and which release containment element portion, and thus the filter element 18, when actuation element 34 is in a second position.

The sheet from which the combined filter sheet 24 is formed may be a continuous sheet, a pre-perforated sheet, or a woven mesh. It may be formed of any material which is sufficiently flexible and compatible with bodily fluids such as blood. Examples of suitable materials include polyurethane, polyolefin, polyester, and silicone polymers. Assembly of the combined filter sheet 24, as well as other structures which make up the filter assembly 10, may employ materials and methods such as adhesives, sewing, solvent welding, ultrasonic welding, crimping, and the like.

In some embodiments, the entire sheet from which the filter and the combination filter sheet 24 is formed is perforated. In other embodiments, the fabrication process punches or drills holes through at least the portion of the sheet which will form the filter element 18 in order to provide fluid communication between the interior and exterior of the filter element 18. In some embodiments, holes associated with the filter are formed at the time that combination filter sheet 24 is extracted from the sheet. In other embodiments, holes are provided in a separate step. Similarly, the fabrication of the combined filter sheet may include forming joining slits 32, generally associated with containment element portion 28, from portions of the original sheet as by perforation, or joining slits 32 may be constructed from different materials and/or in a separate operation.

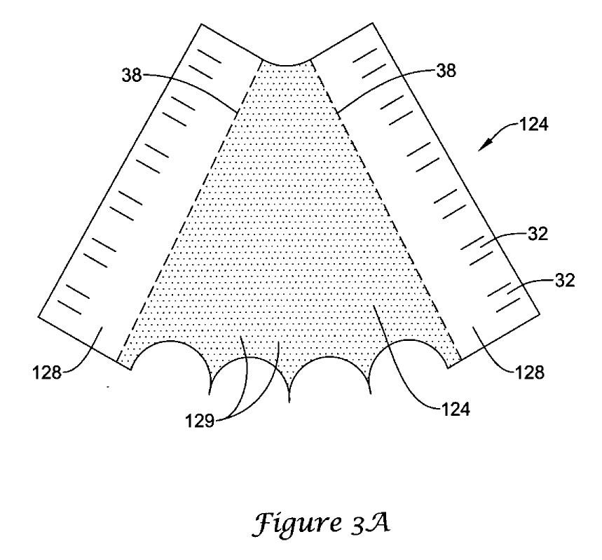

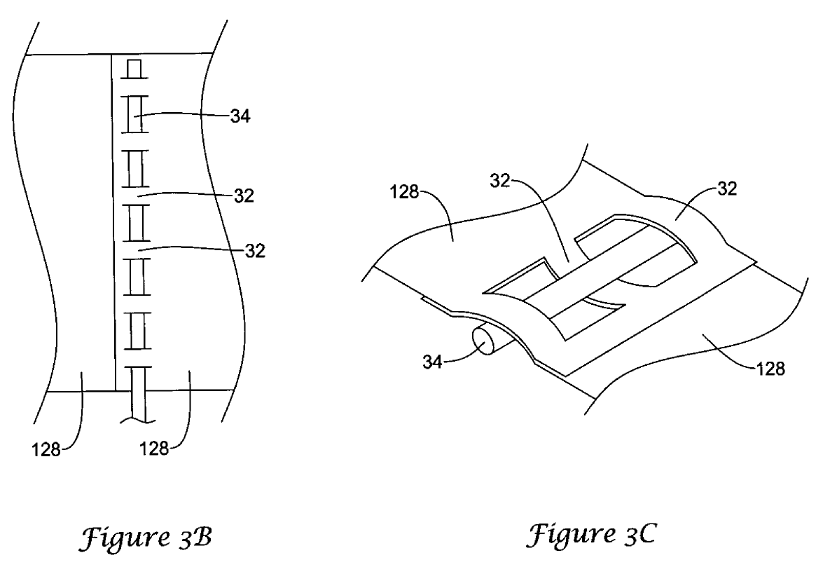

FIG. 3A illustrates a combined filter sheet 124 comprising a filter element portion 124 and two containment elements 128 each of which include joining elements or slits 32. The filter sheet 10 may be cut from a single sheet of appropriate polymer. Holes 129 may be laser cut or punched and should be sized to permit blood cells to pass through freely while retaining emboli and other debris. The generally conical filter assembly is formed by joining the filter sheet along lines 38 by any convenient method such as stitching, gluing, thermal welding and the like. Following attachment to a support structure, such as filter frame 14 of filter assembly 10, the containment element portion 128 are then wrapped about the filter element and/or the associated support structure and joined by inserting actuation element 34 through slits 32 elements as shown in a first position in FIG. 3B and in perspective detail in FIG. 3C. Following positioning the filter assembly in a desired position within a vessel, the actuation element 34 may be withdrawn to a second position to release the joining slits 32 of the containment element portions 24 thereby allowing the filter element 18 to deploy.

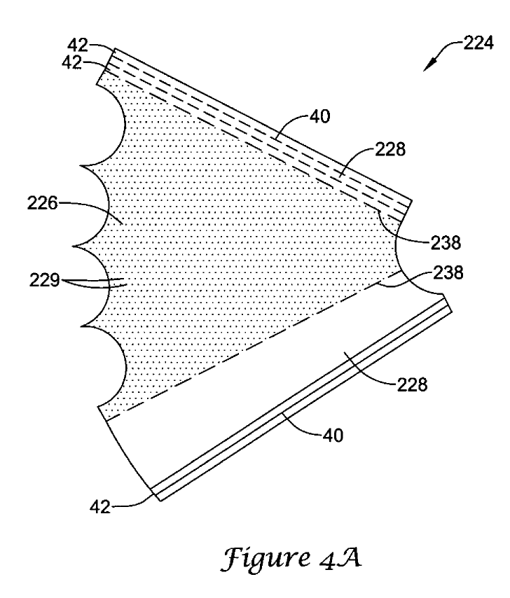

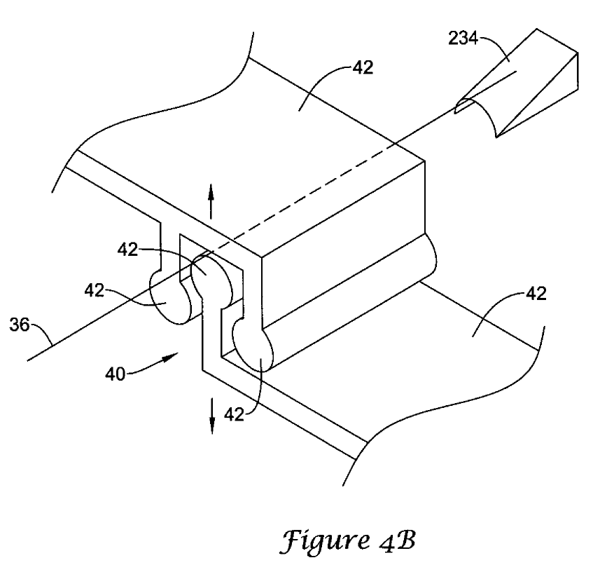

FIG. 4A is a view of an asymmetric combination filter sheet 224 having containment element portions 228 and filter element portion 226 with mating fastener joining elements 40. The joining elements 40 may be attached in a separate operation or, in some embodiments may be thermoformed from a portion of the combination filter sheet 224. In such embodiments, it may be useful to fold the edges of the sheet over to obtain more material from which to form mating structures 40. The sheet 224 is joined along lines 238 as before and the resulting filter is attached to a support structure, for example, filter frame 14. Following collapse of the support structure, the containment element portions 228 are wrapped about filter element 18 and/or the support structure and mating fasteners 40 are pressed together to engage the respective ribs 42 as shown in FIG. 4B. Once the filter has been deployed by a guide wire or catheter, an actuation element 234 having a generally wedge-shaped configuration can be drawn between the two joining elements 40 to separate them in a zipper-like manner thus releasing the filter element.

Various modifications and alterations of this invention will become apparent to those skilled in the art without departing from the scope and principles of this disclosure, and it should be understood that this invention is not to be unduly limited to the illustrative disclosure set forth hereinabove. All publications and patents are herein incorporated by reference to the same extent as if each individual publication or patent was specifically and individually indicated to be incorporated by reference.

What is claimed is:

1. An embolic filter deployment apparatus configured to be used in connection with an intravascular device, the filter deployment apparatus comprising:
a guide wire;
a filter element associated with the guide wire, including a filter element portion;
a containment element portion for the filter element; and
an actuation element for the containment element, movable between a first position and a second position, wherein the filter element portion and the containment element portion are regions of a single sheet.

2. The filter deployment apparatus of claim 1, wherein when the actuation element is in the first position, at least one containment element portion substantially encompasses at least a portion of a filter element and when the actuation element is in the second position, at least one containment element substantially releases at least a portion of a filter element.

3. The filter deployment apparatus of claim 1, wherein the single sheet comprises a sheet or mesh having a plurality of openings therethrough providing fluid communication between at least a portion of its two major surfaces.

4. The filter deployment apparatus of claim 1, wherein the containment element portion includes one or more joining elements which engage the actuation element in at least a first position.

5. The filter deployment apparatus of claim 4, wherein the one or more joining elements include self-mating fasteners.

6. The filter deployment apparatus of claim 1, wherein the containment element portion comprises two or more substantially similar regions of the single sheet of material, exclusive of any joining regions which may be present, separated by the filter element portions of the single sheet of material.

7. The filter deployment apparatus of claim 1, wherein the containment element comprises essentially a single region of the single sheet of material, exclusive of any joining regions which may be present.

8. The filter deployment apparatus of claim 1, wherein at least a portion of the filter element is supported by a support structure.

9. The filter deployment apparatus of claim 1, wherein the support structure is biased to expand the filter element.

10. The filter deployment apparatus of claim 9, wherein when the actuation element is in the first position, at least one containment element portion substantially encompasses at least a portion of the support structure and when the actuation element is in the second position, at least one containment element portion substantially releases at least a portion of the support structure.

11. The filter deployment apparatus of claim 10, wherein when the actuation element is in the second position the support structure is tends to deploy a filter element.

12. A method of deploying an embolic filter comprising:
providing a guide wire;
providing a filter element associated with the guide wire, including a filter element portion;
providing a containment element portion for the filter element, wherein the filter element portion and the containment element are regions of a single sheet;
providing an actuation element, associated with the containment element portion, movable between a first position and a second position;
positioning the filter element in a blood vessel; and
moving the actuation element from a first position to a second position thereby allowing the filter element to deploy from the containment element.

13. The filter deployment apparatus of claim 12, wherein when the actuation element is in the first position, at least one containment element portion substantially encompasses at least a portion of a filter element and when the actuation element is in the second position, at least one containment element portion substantially releases at least a portion of a filter element.

14. The filter deployment apparatus of claim 12, wherein the singlesheet comprises a sheet or mesh having a plurality of openings therethrough providing fluid communication between at least a portion of its two major surfaces.

15. The filter deployment apparatus of claim 12, wherein the containment element portion includes one or more joining elements which engage an actuation element in at least a first position.

16. The filter deployment apparatus of claim 12, wherein the containment element portion comprises two or more substantially similar regions of the single sheet of material, exclusive of any joining regions which may be present, separated by the filter element portions of the single sheet of material.

17. The filter deployment apparatus of claim 12, wherein the containment element portion comprises essentially a single region of the single sheet of material, exclusive of any joining regions which may be present.

18. The filter deployment apparatus of claim 12, wherein at least a portion of the filter element portion is supported by a support structure.

19. The filter deployment apparatus of claim 18, wherein the support structure is biased to expand the filter element.
